(19) United States
(12) Patent Application Publication
(10) Pub. No.: US 2008/0138446 A1
(43) Pub. Date: Jun. 12, 2008

(54) Herbal Composition

(75) Inventor: Karina Anna Hilterman, Victoria (AU)

Correspondence Address: NEEDLE & ROSENBERG, P.C., SUITE 1000, 999 PEACHTREE STREET, ATLANTA, GA, 30309-3915, US

(73) Assignee: LAVENDER HILL PROJECTS PTY LTD, Hawthorn East (AU)

(21) Appl. No.: 11/631,218

(22) PCT Filed: Jul. 2, 2004
(86) PCT No.: PCT/AU04/00895

§ 371 Date: Sep. 4, 2007

(30) Foreign Application Priority Data
Jul. 3, 2003 (NZ) 526850

(51) Int. Cl. A61K 36/00; A61K 36/15; A61K 36/23; A61K 36/268; A61K 36/328; A61K 36/38; A61K 36/50; A61P 17/00; A61K 36/54; A61K 36/66; A61K 36/71; A61K 36/80; A61K 36/86; A61K 36/886

(52) U.S. Cl. 424/726; 424/769; 424/725; 424/757; 424/764; 424/743; 424/737; 424/748; 424/744; 424/754; 424/749; 424/756; 424/730

(57) ABSTRACT

The invention relates to a herbal composition comprising extracts from Thuja, Poke, Sweet violet, Red clover, Greater celandine and Calendula; formulations containing the herbal composition; and use of the herbal composition for preventing and/or treating skin conditions and/or abnormalities.

The invention relates to a herbal composition. Specifically, the invention relates to a herbal composition suitable for treating a variety of skin conditions and/or abnormalities.

### BACKGROUND

The upper skin surface is composed of various layers including the epidermis dermis and subcutaneous. Environmental exposure can cause discolouration, pigmentation and other changes to the upper layers of skin. Changes include age and sun spots, freckles, melanoma, melasma and other skin conditions and/or abnormalities.

Sun spots and other skin discolourations are thought to be caused through cumulative-sun exposure. Areas on the body prone to sun spots include the scalp, neck, face, ears, back of hands and forearms. Once sun spots develop, they do not tend to fade without treatment, regardless of the degree of subsequent sun protection taken.

Freckles are one of the most common skin discolourations amongst the population. Sun-induced freckles are medium-brown in colour, and usually larger and darker than natural freckles. The edges of sun-induced freckles are also irregular in shape, but generally the pigment is even throughout the lesion. Sun-induced freckles have been associated with non-melanoma and melanoma skin tumors.

A melanoma is a malignant tumor that originates in melanocytes or the cells, which produce the pigment melanin that colours skin. The majority of melanomas are black or brown. However, melanomas occasionally stop producing pigment. When this happens, the melanomas may no longer be dark, but are skin-coloured, pink, red, or purple.

Melasma is a skin condition presenting itself as brown patches on the face of adults. Both sides of the face are usually affected. The most common sites of involvement are the cheeks, bridge of nose, forehead, and upper lip. Melasma mostly occurs in women, however men may also be affected. Dark-skinned races tend to have melasma more than others.

Actinic keratoses and solar keratoses are both types of sun-induced skin abnormalities. Both types of keratoses form scaly or crusty bumps on the skin surface. The base of the keratoses may be light or dark, tan, pink, red, or a combination of these colours. Keratosis can produce rough and dry patches on the skin, which can, in turn, become inflamed causing redness to surrounding areas.

Actinic keratoses are often pre-cancerous keratotic lesions, usually caused as a consequence of frequent exposure to the sun. Fair skinned, blonde or red haired people are particularly susceptible to actinic keratoses. Dark skinned people are rarely affected. Actinic keratoses are often pinkish, poorly marginated and often feel scaly or crusty to touch.

Solar keratoses are more frequent amongst the population. Solar keratoses are normally brown in colour and sometimes resemble warts. They are not usually considered to be pre-malignant.

A solar keratosis develops slowly and can generally-reach a size from an eighth to a quarter of an inch. Solar keratoses often appear on the parts of the body are exposed to sunlight such as the face, ears, scalp, neck, back of hands, forearms, shoulders and lips.

If left untreated solar keratoses can grow large and invade the surrounding tissue and, on rare occasions, metastasize to the internal organs. Actinic keratoses are more likely to metastasize to internal organs.

Current orthodox treatments for the above-mentioned skin conditions include cryotherapy (freezing), cauterisation (burning), surgical excision (cutting) or use of esclorotic (eroding) paints, creams and lotions.

The orthodox treatments are invasive, recovery can be painful and usually causes scarring. When using esclorotic agents, care must be taken to avoid contact with healthy-skin as blistering can occur. Therefore, there is a need to provide an improved treatment for treating skin conditions and/or abnormalities or at least to provide the public with a useful, alternative choice of treatment.

### SUMMARY

According to a first aspect of the invention, there is provided a herbal composition comprising extracts from:

Thuja (Thuja occidentalis);

Poke (Phytolacca decandra);

Sweet violet (Viola odorata);

Red clover (Trifolium pratense);

Greater celandine (Chelidonium majus); and

Calendula (Calendula officinalis).

The herbal composition may further comprise extracts of one or more of Blue flag (Iris versicolor), Cleavers (Gallium aparine), Golden seal (Hydrastis canadensis), Sassafras (Sassafras albidum), Yellow dock (Rumex crispus), Echinacea (Echinacea angustifolia or purpurea), Myrrh (Commiphora mol mol), Pasque flower (Anemone pulsatilla), Aloe (Aloe vera), Garlic (Allium sativa), American cranesbill (Geranium maculatum), Figwort (Scrophularia nodosa), Nettles (Urtica dioca), Pilewort (Ranunculus ficaria), Hypericum (Hypericum perforatum), Pumitory (Fumaria officinalis), Slippery elm (Ulmnus fulva), Bloodroot (Sanguinaria canadensis), Burdock (Arctium lappa), Ginger (Zingerber officinalis) and St Mary's Thistle (Silybum marianum).

A second aspect of the invention provides a method of preventing and/or treating a skin condition and/or abnormality which comprises administering a therapeutically effect amount of the herbal composition as defined above to a subject in need thereof.

A third aspect of the invention provides use of the herbal composition as defined above for the manufacture of medicament to prevent and/or treat a skin condition and/or abnormality.

A fourth aspect of the invention provides use of the herbal composition as defined above to prevent and/or treat a skin condition and/or abnormality.

A fifth aspect of the invention provides the herbal composition as defined above for use in preventing and/or treating a skin condition and/or abnormality.

A sixth aspect of the invention provides an agent comprising the herbal composition as defined above for preventing and/or treating a skin condition and/or abnormality.

The herbal composition may conveniently be administered together with one or more carriers. Thus, a seventh aspect of the invention provides the herbal composition as defined above together with one or more carriers.

The herbal composition is suitably administered topically and/or orally.

### DETAILED DESCRIPTION

In the subject specification, except where the context requires otherwise due to express language or necessary implication, the words “comprise” or variations such as “comprises” or “comprising” are used in an inclusive sense, i.e. to specify the presence of the stated features but not to preclude the presence or addition of further features in various embodiments of the invention.

It must be noted that, as used in the subject specification, the singular forms “a”, “an” and “the” include plural aspects unless the context clearly dictates otherwise. Thus, for example, reference to “an extract” includes a single extract, as well as two or more extract; and so forth.

The term “extract” as used herein should be taken in the broadest possible sense. “Extracts” may include tinctures, fluid extracts or solid extracts, tars and infused oils, for example.

The herbal composition may include one or more different kinds of extracts. For example, extracts in the form of a tincture, a tar and an infused oil. Furthermore, the different herbs may be processed to form extracts by different means. Similarly, the herbal composition may comprise a mixture of two or more types of extracts of a particular herb.

Tinctures may be formed from water-based infusions and decoctions. Alternative bases for the extract may include acetracts (to form a vinegar extract), glycetracts (to form a glycerine extract), mels (to form a honey extract), oxymels (honey and vinegar), or syrups (to form a sugar extract).

Tinctures are prepared by methods well known in the art. Briefly, however, a fresh plant tincture is made by first obtaining a herb dried by air as known in the art. The dried herbs are then further dried in an oven at a temperature of between 60° C. and 70° C. The oven dried herbs are then suspended in a solution in a storage container. In a preferred form, the solution is a mixture of alcohol and water. To make up a 1:5 tincture, 75 gms of oven dried herb is suspended in 375 ml of the solution (weight of herb×5 to get 1:5 ratio) in a storage container. The storage container is allowed to stand for about 6 weeks and is shaken periodically. The mixture is then filtered and the solution withdrawn provides the tincture used.

Other methods of extracting herbs are well known in the field. For example, cold percolation techniques may be used to prepare tinctures.

The tinctures may also be purchased from herbal suppliers such as MediHerb Pty Ltd, 124 McEvoy Street, Warwick, Queensland 4370, Australia in a ready-to-use formulation.

An infused oil may be made by infusing or macerating the herb in an oil. Infusing or macerating a herb in an oil solubilises the herb, making the resulting infused oil particularly suitable for topical application.

The oil used to prepare the infused oil may be a fixed or vegetable oil. A fixed oil is a non-volatile animal or plant based oil.

Examples of fixed and vegetable oils include almond oil (including sweet almond oil), coconut oil, corn oil, peanut oil, olive oil (including extra virgin olive oil), safflower oil, sesame oil, soybean oil, castor oil, wheat germ oil and cod-liver oil.

Cold pressed oils are particularly preferred. Methods for preparing cold pressed oils are well known in the field. Generally, the process involves a simple crushing and filtering process.

The process of infusion generally involves the following steps:

allow the herb plant material to wilt over night;

place wilted herb plant material in a container, such as a jar, and cover with the chosen oil;

cover jar with a lid and shake gently to remove any air bubbles around the herb plant material;

remove the lid from the jar and cover the jar with muslin to allow moisture to escape but to prevent foreign objects such as dust and insects to fall into the oil and herb plant material mixture;

leave jar in warm place, covered with a brown paper bag to exclude light, for 2 to 3 weeks;

strain oil and herb plant material mixture through muslin or filter paper into a second jar and leave to stand for week to allow any water from the herb plant material to sink to the bottom;

decant off the oil and leave it to stand again to make sure no moisture remains below the oil;

decant and store infused oil in a cool place in a dark glass vessel.

To make a stronger oil infusion, a fresh lot of herbs is added to the infused oil and the above process is repeated.

Dried herbs may be used in the same way.

This general process of infusion may be used to infuse a variety of oils.

A tar is a very strong decoction, which is further reduced by evaporation.

The extracts may be prepared from any part of the herb plant such as, for example, foliage, leaves, the root, flowers, bark, stems or rhizome, seeds, and fruit. However, particular parts of the herb plant are usually used to prepare the extracts.

A Thuja extract is usually made from the foliage. In a preferred embodiment of the invention, the Thuja extract is prepared as a tincture. The herbal composition preferably comprises from about 0.5 to about 20% of Thuja extract, more preferably from about 6 to about 10% of Thuja extract, and most preferably about 8% of Thuja extract.

A Poke extract is usually made from the root. In a preferred embodiment of the invention, the Poke extract is prepared as a tincture. The herbal composition preferably comprises from about 1 to about 20% of Poke extract, more preferably from about 14 to about 18% of Poke extract, and most preferably about 16% of Poke extract.

A Sweet violet extract is usually made from the leaves. In a preferred embodiment of the invention, the Sweet violet extract is prepared as a tincture. The herbal composition preferably comprises from about 0.5 to about 12% of Sweet violet extract, more preferably from about 6 to about 10% of Sweet violet extract, and most preferably about 8% of Sweet violet extract.

A Red clover extract is usually made from the flowers. In a preferred embodiment of the invention, the Red clover extract is prepared as a tar. When the Red clover extract is in the form of a tar, the herbal composition preferably comprises from about 1 to about 30% of Red clover extract, more preferably from about 14 to about 18% of Red clover extract, and most preferably about 16% of Red clover extract. The Red clover extract may also be in the form a tincture. When this herb extract is in the form of a tincture, the herbal composition preferably comprises from about 0.5 to about 20% of Red clover extract, more preferably from about 2 to about 6% of Red clover extract, and most preferably about 4% of Red clover extract.

A Greater celandine extract is usually made from the foliage. In a preferred embodiment of the invention, the Greater celandine extract is prepared as a tincture. The herbal composition preferably comprises from about 0.2 to about 10% of Greater celandine extract, more preferably from about 3 to about 4% of Greater celandine extract, and most preferably about 2% of Greater celandine extract.

A Calendula extract is usually made from the flowers. In a preferred form of the invention, the Calendula extract is infused in an oil. However, the Calendula extract may also be prepared as a tincture. The herbal composition preferably comprises from about 1 to about 16% of Calendula extract, more preferably from about 6 to about 10% of Calendula extract, and most preferably about 8% of Calendula extract.

Table 1 illustrates a herbal composition of a preferred embodiment of the invention.

TABLE 1

| Common Name | Approved Name | Herb Part | Preparation |
|---|---|---|---|
| Thuja | Thuja occidentalis | Foliage | Tincture |
| Poke | Phytolacca decandra | Root | Tincture |
| Sweet violet | Viola odorata | Leaves | Tincture |
| Red clover | Trifolium pratense | Flowers | Tar |
| Greater celandine | Chelidonium majus | Foliage | Tincture |
| Calendula | Calendula officinalis | Flowers | Infused oil |

In addition to the above-mentioned herb extracts, the herbal composition may further comprise one or more additional herb extracts, which may also assist in preventing and/or treating a skin condition and/or abnormality. Examples include herb extracts of one or more of Blue flag (Iris vesicular), Cleavers (Gallium aparine), Golden seal (Hydrastis canadensis), Sassafras (Sassafras albidum), Yellow dock (Rumex crispus), Echinacea (Echinacea angustifolia or purpurea), Myrrh (Commiphora mol mol), Pasque flower (Anemone pulsatilla), Aloe (Aloe vera), Garlic (Allium sativa), American cranesbill (Geranium maculatum), Figwort (Scrophularia nodosa), Nettles (Urtica dioca), Pilewort (Ranunculus ficaria), Hypericum (Hypericum perforatum), Fumitory (Fumaria officinalis), Slippery elm (Ulmnus fulva), Bloodroot (Sanguinaria canadensis), Burdock (Arctium lappa), Ginger (zingerber officinalis) and St Mary's Thistle (Silybum marianum).

The herbal composition may comprise from about 0.1% to about 30%, and more preferably from about 0.1% to about 10%, of each of the above-mentioned additional herb extracts.

The skin conditions and/or abnormalities include skin pigmentation disorders and benign and malignant skin growths.

Skin pigmentation disorders are conditions that cause the skin to appear lighter or darker than normally natural, or blotchy and irregular. Skin pigmentation disorders may be present at birth or develop later in life. Skin pigmentation disorders may also be sun-induced. Examples of skin pigmentation disorders include, but are not limited to, age and sun spots; freckles; pigment loss after sun damage; melasma; and discolouration.

Skin abnormalities include benign and malignant skin growths. Examples of benign and malignant skin growths include, but are not limited to, skin cancers or tumors such as basal cell carcinoma, squamous cell carcinoma, malignant melanoma; actinic keratoses and solar keratoses; skin lesions; and irregular skin cell development. Any one of these skin abnormalities may be pigmented or non-pigmented.

Not wishing to be bound by a particular theory or hypothesis the inventor believes that the surprising and unexpected effects seen with the herbal composition results from synergism between the individual components. As each of the above-mentioned additional herb extracts has its own individual therapeutic action, the synergy between the individual components in the herbal composition can be further enhanced by addition of one or more of these additional herb extracts in the herbal composition.

The term “subject” as used herein refers to any animal having symptoms associated with or caused by viral infections, which requires treatment with the herbal composition. The subject may be an animal, such as a mammal, preferably a human, or may be a non-human primate or non-primates such as used in animal model testing. While it is particularly contemplated that the herbal composition is suitable for use in medical treatment of humans, it is also applicable to veterinary treatment, including treatment of companion animals such as dogs and cats, and domestic animals such as horses, ponies, donkeys, mules, llama, alpaca, pigs, cattle and sheep, or zoo animals such as primates, felids, canids, bovids, and ungulates.

Generally, the terms “treating”, “treatment” and the like are used herein to mean affecting a subject, tissue or cell to obtain a desired pharmacological and/or physiological effect. The effect may be prophylactic in terms of completely or partially preventing one or more skin conditions and/or abnormalities, and/or may be therapeutic in terms of a partial or complete cure of one or more skin conditions and/or abnormalities. “Treating” as used herein covers any prevention or treatment of a skin condition and/or abnormality in an animal such as a mammal, more particularly a human, and includes:

(a) preventing one or more of the skin conditions and/or abnormalities referred to above from occurring in a subject that may be predisposed to such symptoms, but has not yet developed the skin condition and/or abnormality;
(b) inhibiting the development of one or more of the skin conditions and/or abnormalities referred to above; or
(c) alleviating, relieving or ameliorating the effects, i.e. cause regression, of one or more of the skin conditions and/or abnormalities referred to above.

As used herein, the term “effective amount” means an amount of the herbal composition to treat and/or prevent one or more of the skin conditions and/or abnormalities referred to above in order to yield a desired therapeutic response.

The term “therapeutically effective amount” means an amount of the herbal composition to yield a desired therapeutic response. For example, preventing and/or treating one or more of the skin conditions and/or abnormalities referred to above.

The herbal composition may conveniently be administered together with one or more carriers. Carriers include substances that are useful in preparing a formulation comprising the herbal composition, may be in co-administration with the herbal composition while allowing the individual components to perform their intended function, and are generally safe, non-toxic and are neither biologically or otherwise undesirable. In addition, carriers will include those suitable for veterinarian use as well as human use. Examples of carriers include dispersing agents, suspending agents, emulsifying agents, stabilising agents, wetting agents, binding agents, lubricants, disintegrants, solvents, media, delay agents, fillers, aqueous and oily bases, non-aqueous vehicles, i.e. edible oils, and the like.

In addition, the herbal composition may contain preserving agents, sweetening agents, colouring agents, flavouring agents, thickening and/or gelling agents, buffers and the like. Those skilled in the art will be able to identify further additives that may be desirable for cosmetic reasons, palatability, or shelf life, for example.

As it will be appreciated by those skilled in the art to which the invention relates, the herbal composition may be converted into customary formulations. Examples of formulations include, but are not limited to, solutions, emulsions, suspensions, powders, granules, natural and synthetic materials impregnated with the individual components of the herbal composition, pills, capsules, tablets, cachets, pastilles, lozenges, bolus, electuary, pastes, ointments, creams, plasters, washes, lotions, transdermal patches, enemas, suppositories, pessaries, sprays (atomiser, or aerosol) mouthwashes, syrups, and/or elixirs. In a preferred form of the invention, the herbal composition is converted into a formulation suitable for topical administration.

The choice of carriers and/or additives may be dictated to some extent by the intended dosage form of the herbal composition and the mode of administration of the herbal composition.

Formulations comprising the herbal composition may be produced by a number of techniques standard in the art, for example, by mixing the herb extracts with one or more carriers and/or additives until all components are well blended. The formulations may be stored in suitable containers, receptacles, vessels and the like.

Formulations suitable for oral administration may conveniently be presented in discrete units such as capsules, cachets or tablets each containing a predetermined amount of the herbal composition; as a powder or granules; as a solution, a suspension or as an emulsion. The herbal composition may also be presented as a bolus, electuary or paste. Tablets and capsules for oral administration may contain conventional carriers such as binding agents, fillers, lubricants, disintegrants, or wetting agents. The tablets may be coated according to methods well known in the art. Oral liquid preparations may for example be in the form of aqueous or oily suspensions, emulsions, syrups or elixirs, or may be presented as a dry product, such as a powder or granules, for constitution with water or another suitable vehicle, e.g. orange juice, before use. Suitable carriers for such liquid preparations may include, for example, ethyl alcohol, vinegars, glycerine, honey, sugar, or water. Such liquid preparations may also contain conventional additives such as suspending agents, emulsifying agents, non-aqueous vehicles, which may include edible oils, or preservatives.

For topical administration to the skin, the herbal composition may be formulated as ointments, creams, compress, poultice, moisturiser, plasters, washes, lotions, or as a transdermal patch. Ointments and creams may, for example, be formulated with an aqueous or oily base with the addition of suitable thickening and/or gelling agents. Examples of oily bases include, but are not limited to, a vitamin E cream or lotion, glucate cream, extra virgin olive oil, sweet almond oil, jojoba oil, apricot oil, peanut oil, fruit and vegetable oils, beeswax, lanolin and honey. Lotions and washes may be formulated with an aqueous or oily base, and will in general also contain one or more emulsifying agents, stabilising agents, dispersing agents, suspending agents, thickening agents, or colouring agents.

A topical formulation in the form of a cream may be prepared by adding about 40 mls of Thuja extract in the form of a tincture; about 80 mls of Poke extract in the form of a tincture; about 40 mls of Sweet violet extract in the form of a tincture; about 20 mls of Red clover extract in the form of a tincture and about 80 mls of Red clover extract in the form of a tar; about 10 mls of Greater celandine extract in the form of a tincture; and about 10 mls of Calendula extract in the form of an infused oil to about 200 mls of a suitable cream base (approximately about 40%). These ingredients may be blended by mixing, agitation and/or heating, as known in the art. The cream based topical formulation may then be apportioned into suitable containers for use.

Formulations suitable for topical administration in the mouth include lozenges comprising the herbal composition and may have a flavoured base such as sucrose and gum acacia or gum tragacanth; pastilles comprising the herbal composition in an inert base such as gelatin or sucrose and gam acacia; and mouthwash in a suitable liquid carrier.

In use, the herbal composition would be administered to a subject as needed, as desired, or as advised by a medical practitioner, pharmacist, attendant physician, medical herbalist, naturopath, or veterinarian. The selection of the herb extracts may also be made on the specific needs of the subject.

As will be appreciated, the dose administered, the period of administration, and the general administration regime may differ between subjects depending on variables such as their tolerance to certain active ingredients, weight, metabolism, the mode of administration chosen, the severity of the symptoms, and the age and/or general health of the subject. In general, when the herbal composition is converted into a formulation suitable for topical administration, the general administration regime for topical treatment is application to the affected areas about 3 times daily. Topical treatment may also be supplemented with an oral administration.

Treatment is preferably commenced before or at the time the skin condition and/or abnormality develops and preferably continues until the skin condition and/or abnormality is no longer present.

The invention will now be further elucidated by reference to the following non-limiting examples.

### EXAMPLES

### Subject A

### Personal Details:

Female, in her late 40s, with blonde hair, blue eyes and fair skin. She had much exposure to the sun during adolescence and early adulthood.

### Condition(s):

Age spots had developed on the back of hands, forearms and forehead. Some of the age spots had become itchy and scaly.

### Recommended Orthodox Treatment:

Removal by cauterisation (burning).

### Subject B

### Personal Details:

Female, in her late 60s, with very fair skin. She was well-tanned due to much exposure to the sun through a very active outdoor life.

### Condition(s):

Developed a proliferation of sun-induced skin abnormalities including actinic and solar keratoses and basal cell carcinomas as well as sun spots.

### Recommended Orthodox Treatment:

Removal by cauterisation (burning).

### Subject C

### Personal Details:

Female, in her mid 70s, with fair skin. She had a very active outdoor life.

### Condition(s):

Developed a proliferation of sun-induced skin abnormalities including actinic and solar keratoses and basal cell carcinomas as well as sun spots.

### Recommended Orthodox Treatment:

Removal by cauterisation (burning).

### Subject D

### Personal Details:

Male, in his mid 80s, with a fair complexion.

### Condition(s):

Developed a melanoma on the side of his nose.

### Recommended Orthodox Treatment:

Surgical excision (cutting).

### Subject E

### Personal Details:

Female, in her early 40s, with blonde hair and olive complexion. She had a very active outdoor life.

### Condition(s):

Developed sun spots on her face.

### Recommended Orthodox Treatment:

No orthodox treatment sought.

### Subject F

### Personal Details:

Male, in his late 60s, with a fair complexion. He lived an outdoor rural life.

### Condition(s):

Developed sun spots and actinic keratoses.

### Recommended Orthodox Treatment:

Surgical excision (cutting) was recommended due to a rapidly changing actinic keratoses on his nose.

### Subject G

### Personal Details:

Male, in his mid 50s, with a very fair complexion. He had worked outdoors for most of his adult life.

### Condition(s):

12 year history of basal cell carcinomas and two squamous cell carcinomas.

### Recommended Conventional Treatment:

Surgical excision (cutting), removal by cauterisation (burning) and/or use of an esclorotic (eroding) cream.

### Treatment

Each subject was treated with a herbal composition according to the invention.

The herbal composition was prepared as a topical formulation in the form of a cream by mixing the following ingredients:

about 40 mls of Thuja extract in the form of a tincture;

about 80 mls of Poke extract in the form of a tincture;

about 40 mls of Sweet violet extract in the form of a tincture;

about 20 mls of Red clover extract in the form of a tincture and about 80 mls of Red clover tar;

about 10 mls of Greater celandine extract in the form of a tincture;

about 10 mls of Calendula extract in the form of an infused oil; and

about 200 mls of a glucate cream base comprising sweet almond oil, glucose and water.

Each subject was required to apply the cream at least 3 times daily to the affected areas.

### Results

Subject A applied the cream as required for a period of 3 months. At the end of this period, all signs of the age spots had disappeared, leaving normal, healthy skin, i.e. no scarring.

Subject B applied the cream as required. The skin abnormalities and sun spots disappeared, leaving normal, healthy skin, i.e. no scarring. There were no signs of any new skin abnormalities developing.

Subject C applied the cream as required. The skin abnormalities and sun spots disappeared, leaving healthy skin in its place, i.e. no scarring. Continued use of the cream for a period of time prevented new sun spots from developing.

Subject D applied the cream as required for a two week period. After this time, the melanoma shriveled and dropped off, leaving healthy, normal skin in its place, i.e. no scarring. There have been no reported reoccurrence of melanomas.

Subject E applied the cream as required. After a period of 1 month, the sun spots had disappeared, leaving normal, healthy skin, i.e. no scarring.

Subject F applied the cream as required, and within 1 month of treatment, the sun spots and actinic keratoses had disappeared, leaving normal, healthy skin, i.e. no scarring.

Subject G applied the cream as required. Within a short period of time, the skin abnormalities had disappeared.

Wherein the foregoing description reference has been made to specific component or integers of the invention which known equivalents then such equivalents are herein incorporated as if individually set forth.

Although this invention has been described by example and with reference to possible embodiment thereof, it is to be understood that modifications or improvements may be made thereto without departing from the scope of the invention.

1-46. (canceled)

47. A herbal composition comprising extracts from:
Thuja;
Poke;
Sweet violet;
Red clover;
Greater celandine; and
Calendula.

48. A herbal composition according to claim 47, wherein the herbal composition comprises from about 0.5 to about 20% of Thuja extract.

49. A herbal composition according to claim 47, wherein the herbal composition comprises from about 1 to about 20% of Poke extract.

50. A herbal composition according to claim 47, wherein the herbal composition comprises from about 0.5 to about 12% of Sweet violet extract.

51. A herbal composition according to claim 47, wherein the herbal composition comprises from about 1 to about 30% of Red clover extract when the Red clover extract is in the form of a tar and/or from about 0.5 to about 20% of Red clover extract when the Red clover extract is in the form of a tincture.

52. A herbal composition according to claim 47, wherein the herbal composition comprises from about 0.2 to about 10% of Greater celandine extract.

53. A herbal composition according to claim 47, wherein the herbal composition comprises from about 1 to about 16% of Calendula extract.

54. A herbal composition according to claim 47, wherein the herbal composition comprises from about 6 to about 10% of Thuja extract.

55. A herbal composition according to claim 47, wherein the herbal composition comprises from about 14 to about 18% of Poke extract.

56. A herbal composition according to claim 47, wherein the herbal composition comprises from about 6 to about 10% of Sweet violet extract.

57. A herbal composition according to claim 47, wherein the herbal composition comprises from about 14 to about 18% of Red clover extract when the Red clover extract is in the form of a tar and/or from about 2 to about 6% of Red clover extract when the Red clover extract is in the form of a tincture.

58. A herbal composition according to claim 47, wherein the herbal composition comprises from about 3 to about 4% of Greater celandine extract.

59. A herbal composition according to claim 47, wherein the herbal composition comprises from about 6 to about 10% of Calendula extract.

60. A herbal composition according to claim 47, wherein the herbal composition comprises about 8% of Thuja extract.

61. A herbal composition according to claim 47, wherein the herbal composition comprises about 16% of Poke extract.

62. A herbal composition according to claim 47, wherein the herbal composition comprises about 8% of Sweet violet extract.

63. A herbal composition according to claim 47, wherein the herbal composition comprises about 16% of Red clover extract when the Red clover extract is in the form of a tar and/or about 4% of Red clover extract when the Red clover extract is in the form of a tincture.

64. A herbal composition according to claim 47, wherein the herbal composition comprises about 2% of Greater celandine extract.

65. A herbal composition according to claim 47, wherein the herbal composition comprises about 8% of Calendula extract.

66. A herbal composition according to claim 47, wherein the herbal composition further comprises extracts of one or more of Blue flag, Cleavers, Golden seal, Sassafras, Yellow dock, Echinacea, Myrrh, Pasque flower, Aloe, Garlic, American cranesbill, Figwort, Nettles, Pilewort, Hypericum, Fumitory, Slippery elm, Bloodroot, Burdock, Ginger and St Mary's Thistle.

67. A herbal composition according to claim 47, wherein the herbal composition comprises from about 0.1% to about 30% of extracts of one or more of Blue flag, Cleavers, Golden seal, Sassafras, Yellow dock, Echinacea, Myrrh, Pasque flower, Aloe, Garlic, American cranesbill, Figwort, Nettles, Pilewort, Hypericum, Fumitory, Slippery elm, Bloodroot, Burdock, Ginger and St Mary's Thistle.

68. A herbal composition according to claim 47, wherein the herbal composition comprises from about 0.1% to about 10% of extracts of one or more of Blue flag, Cleavers, Golden seal, Sassafras, Yellow dock, Echinacea, Myrrh, Pasque flower, Aloe, Garlic, American cranesbill, Figwort, Nettles, Pilewort, Hypericum, Fumitory, Slippery elm, Bloodroot, Burdock, Ginger and St Mary's Thistle.

69. A herbal composition according to claim 47, wherein the extract is in the form of a tincture, a fluid extract or a solid extract, a tar, an infused oil, or a mixture thereof.

70. A herbal composition according to claim 47, wherein the Thuja extract is in the form of a tincture, the Poke extract is in the form of a tincture, the Sweet violet extract is in the form of a tincture, the Red clover extract is in the form of a tar, the Greater celandine extract is in the form of a tincture and the Calendula extract is infused in an oil or is in the form of a tincture.

71. A herbal composition according to claim 47, further comprising Red clover extract in the form of a tincture.

72. A herbal formulation comprising a herbal composition according to claim 47.

73. A herbal formulation according to claim 72, wherein the herbal formulation is in the form of a topical formulation.

74. A herbal formulation according to claim 73, wherein the topical formulation is a cream.

75. A herbal formulation according to claim 72, wherein the herbal formulation is in the form of an oral formulation.

76. A herbal formulation according to claim 75, wherein the oral formulation is a solution.

77. A method of preventing and/or treating a skin condition and/or abnormality which comprises administering a therapeutically effect amount of a herbal composition according to claim 47.

78. A method according to claim 77, wherein the skin condition is a skin pigmentation disorder.

79. A method according to claim 78, wherein skin pigmentation disorder is an age or sun spots; freckle; pigment loss after sun damage; melasma; or discolouration.

80. A method according to claim 77, wherein the skin abnormality is a benign or malignant skin growth.

81. A method according to claim 80, wherein the benign or malignant skin growth is a skin cancer or tumor; actinic keratosis; solar keratosis; a skin lesion; or an irregular skin cell development.

82. A method according to claim 81, wherein the skin cancer or tumor is a basal cell carcinoma; squamous cell carcinoma; or malignant melanoma.

83. A method according to claim 82, wherein the benign or malignant skin growth is pigmented or non-pigmented.

84. A method according to claim 83, wherein the subject is an animal, mammal or human.

85. A method according to claim 84, wherein the herbal composition or herbal formulation is in the form of a topical formulation.

86. A method according to claim 85, wherein the topical formulation is in the form of a cream.

87. A method according to claim 86, wherein the cream is applied about 3 times daily.

88. A method according to claim 88, wherein the herbal composition or herbal formulation is administered topically and/or orally.
